(19) United States
(12) Patent Application Publication
(10) Pub. No.: US 2021/0402143 A1
(43) Pub. Date: Dec. 30, 2021

(54) CATHETER ASSEMBLY

(71) Applicant: TERUMO KABUSHIKI KAISHA, Tokyo (JP)

(72) Inventors: Takayuki YOKOTA, Chuo-shi (JP); Shuichi KIMURA, Inagi-shi (JP)

(73) Assignee: TERUMO KABUSHIKI KAISHA, Tokyo (JP)

(21) Appl. No.: 17/474,923

(22) Filed: Sep. 14, 2021

Related U.S. Application Data
(63) Continuation of application No. PCT/JP2020/010683, filed on Mar. 12, 2020.

(30) Foreign Application Priority Data
Mar. 18, 2019 (JP) 2019-050068

(51) Int. Cl. A61M 25/00; A61M 25/06; A61M 39/22

(52) U.S. Cl. CPC A61M 25/0097; A61M 39/22; A61M 25/06

(57) ABSTRACT

A catheter assembly includes: a catheter having a lumen; a catheter hub located at a proximal end of the catheter and having a hollow portion that communicates with the lumen; and a valve body located in the catheter hub, wherein the valve body is an monolithic structure including: a fixed portion fixed to the catheter hub, a deformable portion continuous with the fixed portion and having a slit that is openable and closable, and a pusher portion extending from the deformable portion in a proximal direction. The pusher portion is configured to be pushed in a distal direction by a connector inserted into the hollow portion from a proximal end of the catheter hub to deform the deformable portion and open the slit.

### CROSS-REFERENCE TO RELATED APPLICATIONS

This application is a bypass continuation of PCT Application No. PCT/JP2020/010683, filed on Mar. 12, 2020, which claims priority to Japanese Application No. 2019-050068, filed on Mar. 18, 2019. The contents of these applications are hereby incorporated by reference in their entireties.

### BACKGROUND

The present disclosure relates to a catheter assembly having a valve body in a catheter hub that inhibits blood from flowing out.

A catheter assembly that functions as an indwelling needle is used when performing a medicinal fluid infusion and a blood transfusion. For example, a catheter assembly disclosed in JP 2014-528808 A includes a catheter and a catheter hub (catheter adapter) fixed to the catheter. The catheter assembly is inserted into a patient with an introducer needle housed in the catheter during use, and the introducer needle is detached from the catheter and catheter hub after being inserted. Then, the catheter assembly serves as an inlet/outlet for a medicinal liquid or blood as a connector of a medical device (medicinal liquid infusion or blood transfusion tube) is inserted into the catheter hub after detachment.

In addition, the catheter assembly disclosed in JP 2014-528808 A includes, in the catheter hub, a valve body (septum) that inhibits blood leakage during indwelling, and an opening member (septum actuator) configured to open the valve body. When the connector is connected, the connector is inserted into the catheter hub to push the opening member, so that a slit of the valve body is open and the liquid can flow.

### SUMMARY

However, when the catheter assembly disclosed in JP 2014-528808 A is manufactured, a plurality of components (the valve body and the opening member) are incorporated in the catheter hub. Thus, the catheter assembly has a disadvantage that the work of assembling the respective components requires manufacturing time, and manufacturing cost increases as the number of components and manufacturing time increase.

Embodiments of the present disclosure have been developed to solve the above-described problems, and an object thereof is to provide a catheter assembly capable of reducing manufacturing time and manufacturing cost by reducing the number of components with a simple configuration.

According to one embodiment, a catheter assembly includes: a catheter having a lumen; a catheter hub provided at a proximal end of the catheter and having a hollow portion that communicates with the lumen; and a valve body provided in the catheter hub. The valve body is an monolithic structure including: a fixed portion fixed to the catheter hub; a deformable portion continuous with the fixed portion and having a slit that is openable and closable; and a pusher portion extending from the deformable portion in a proximal direction. The pusher portion is pushed in a distal direction by a connector, inserted into the hollow portion from a proximal end of the catheter hub, to deform the deformable portion and open the slit.

In the above catheter assembly, the deformable portion is deformed and the slit can be favorably open when the pusher portion is displaced with the insertion of the connector by the valve body, which is the monolithic structure of the fixed portion, the deformable portion, and the pusher portion. As a result, the liquid flowing from the connector is smoothly guided to the distal side of the valve body through the slit. Therefore, the catheter assembly can reduce the number of components with a simple configuration without requiring the conventional opening member, which makes it possible to reduce manufacturing time and manufacturing cost.

### BRIEF DESCRIPTION OF THE DRAWINGS

FIG. 1 is a perspective view illustrating an overall configuration of a catheter assembly according to a first embodiment of the present invention;

FIG. 2 is a side cross-sectional view illustrating the inside of a catheter hub of FIG. 1;

FIG. 3 is a perspective view illustrating a valve body housed in the catheter hub;

FIG. 4 is a side cross-sectional view illustrating an operation when a connector is inserted in the catheter hub of FIG. 2;

FIG. 5 is a perspective view illustrating a valve body housed in a catheter assembly according to a second embodiment of the present invention;

FIG. 6A is a side cross-sectional view illustrating the inside of a catheter hub housing the valve body of FIG. 5, and FIG. 6B is a side cross-sectional view illustrating an operation when a connector is inserted in the catheter hub of FIG. 6A;

FIG. 7 is a perspective view illustrating a valve body housed in a catheter assembly according to a third embodiment of the present invention;

FIG. 8A is a side cross-sectional view illustrating the inside of a catheter hub housing the valve body of FIG. 7, and

FIG. 8B is a side cross-sectional view illustrating an operation when a connector is inserted in the catheter hub of FIG. 8A;

FIG. 9 is a perspective view illustrating a valve body housed in a catheter assembly according to a fourth embodiment of the present invention; and

FIG. 10A is a side cross-sectional view illustrating the inside of a catheter hub housing the valve body of FIG. 9, and FIG. 10B is a side cross-sectional view illustrating an operation when a connector is inserted in the catheter hub of FIG. 10A.

### DETAILED DESCRIPTION

Hereinafter, preferred embodiments of the present invention will be described in detail with reference to the accompanying drawings.

### First Embodiment

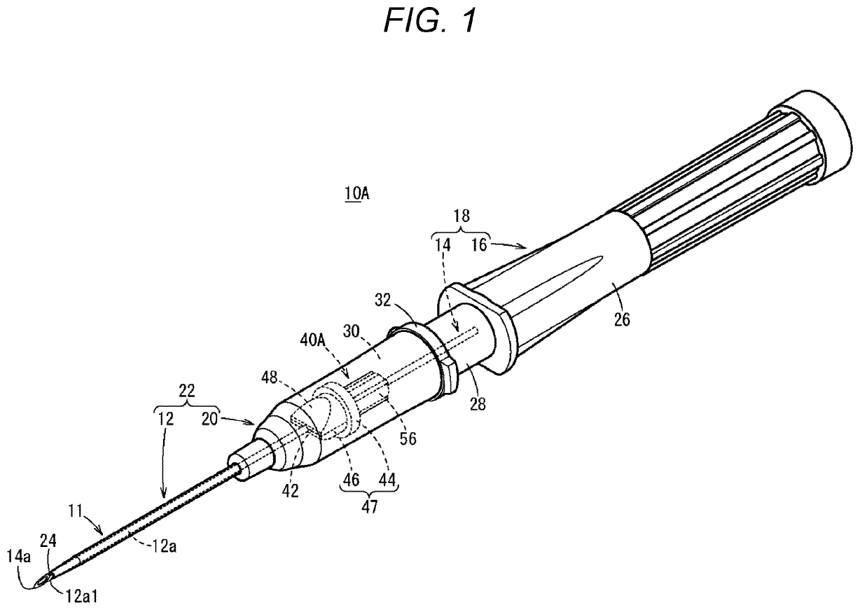

A catheter assembly 10A according to a first embodiment of the present invention has a catheter 12 that is inserted to indwell inside a patient's body (living body) as illustrated in FIG. 1, and is used to construct an inlet/outlet for a liquid (a medicinal liquid and blood) during a medicinal liquid infusion, a blood transfusion, or the like. The catheter 12 is configured as a peripheral venous catheter. Incidentally, the catheter 12 may be a catheter longer than the peripheral venous catheter (for example, a central venous catheter). In addition, the catheter 12 is not limited to a venous catheter, and may be configured as an arterial catheter such as a peripheral arterial catheter.

As illustrated in FIG. 1, the catheter assembly 10A has an operating body 18 configured of an inner needle 14 and a needle hub 16 fixed to a proximal end of the inner needle 14. In addition, the catheter assembly 10A has a catheter indwelling body 22 configured of the above-described catheter 12 and a catheter hub 20 fixed to a proximal end of the catheter 12.

In the catheter assembly 10A, a multi-structure needle 11 in which the inner needle 14 is inserted into the catheter 12 is formed as the operating body 18 is assembled to a proximal end of the catheter indwelling body 22 in an initial state before use (product provided state). In the multi-structure needle 11, the needle tip 14a of the inner needle 14 protrudes from a distal end of the catheter 12, and the inner needle 14 and the catheter 12 can be integrally punctured into the patient.

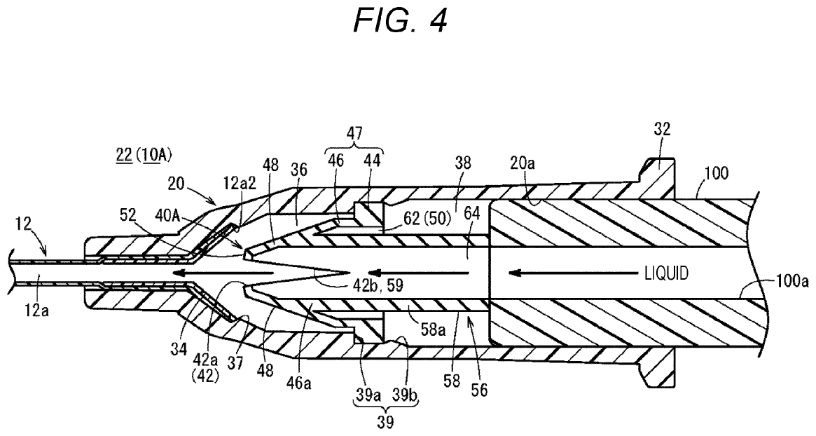

When using the catheter assembly 10A, a user, such as a doctor and a nurse, grips and operates the needle hub 16 to puncture the multi-structure needle 11 into the patient's body, thereby setting a puncture state in which the needle tip 14a reaches a blood vessel. Further, the user inserts the catheter 12 into the blood vessel by advancing the catheter 12 relative to the inner needle 14 while maintaining the puncture state. Then, the catheter indwelling body 22 indwells in the patient by retracting the inner needle 14 relative to the catheter 12 and further removing the inner needle 14 from the catheter hub 20. Then, the catheter indwelling body 22 can perform treatment such as administration of a medicinal liquid or blood to a patient and blood collection from the patient by connecting a connector 100 of a medical device (see FIG. 4) to the catheter hub 20. Hereinafter, each configuration of the catheter assembly 10A will be described in detail.

The inner needle 14 of the catheter assembly 10A (the operating body 18) is configured as a hollow tube (or a solid rod) having rigidity capable of puncturing a skin of a living body, and has the sharp needle tip 14a at a distal end thereof. An outer peripheral surface of the inner needle 14 is provided with a groove 24 for flashback that guides blood to the proximal side when puncturing the blood vessel. Incidentally, the structure for performing flashback is not particularly limited, and for example, a configuration having a hole (not illustrated) communicating with an inner space of the inner needle 14 on the proximal side of the distal end of the catheter 12 may be provided.

Examples of a constituent material of the inner needle 14 include a metal material such as stainless steel, aluminum or an aluminum alloy, and titanium or a titanium alloy, a hard resin, ceramics, and the like. The inner needle 14 is firmly fixed to the needle hub 16 by an appropriate fixing means such as fusion, adhesion, and insert molding.

The needle hub 16 forms a grip portion to be gripped by the user in the initial state in which the catheter indwelling body 22 and the operating body 18 are assembled. The needle hub 16 includes a hub main body 26 gripped by the user and an inner needle support portion 28 monolithicly molded at a distal end of the hub main body 26. The hub main body 26 is formed into a cylindrical shape on the proximal side, and is gradually changes into a square tube shape toward the distal side. The inner needle support portion 28 is formed into a columnar shape protruding from the hub main body 26 in the distal direction, and holds a proximal portion of the inner needle 14 at the central portion thereof.

A constituent material of the needle hub 16 is not particularly limited, but a thermoplastic resin, such as polypropylene, polycarbonate, polyamide, polysulfone, polyarylate, and a methacrylate-butylene-styrene copolymer can be applied.

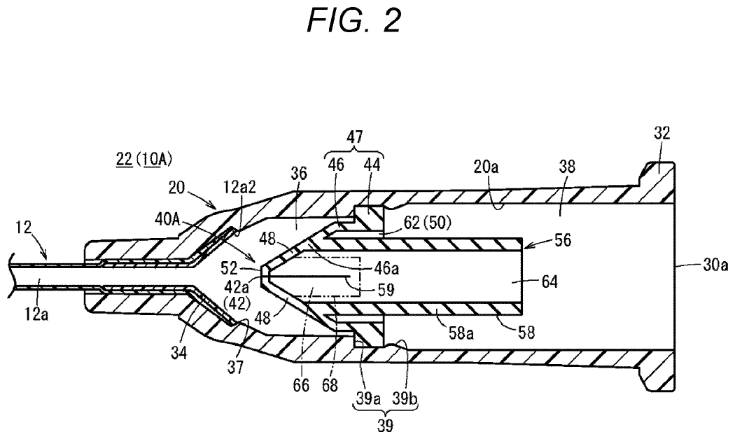

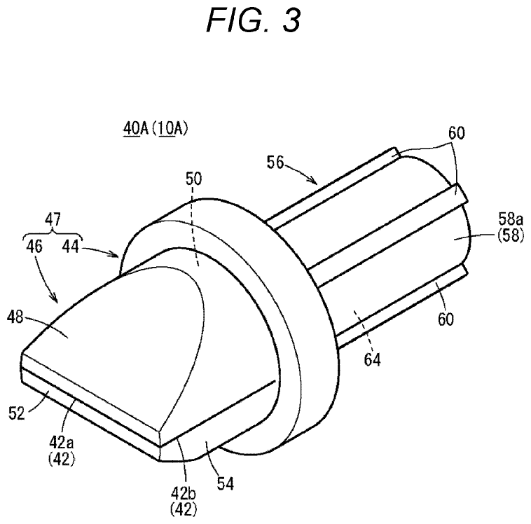

On the other hand, the catheter 12 of the catheter assembly 10A is configured as a flexible hollow tube in which a lumen 12a is formed inside. An outer shape of the catheter 12 and the lumen 12a are formed into a perfect circular shape in a cross-sectional view orthogonal to the axial direction, and extend along the axial direction of the catheter 12. The lumen 12a communicates with a distal opening 12a1 formed at a distal end of the catheter 12 and a proximal opening 12a2 (see FIG. 2) formed at a proximal end of the catheter 12.

A material forming the catheter 12 is not particularly limited, but a transparent soft resin material may be applied. Examples of a constituent material of the catheter 12 include a fluorine-based resin such as polytetrafluoroethylene (PTFE), an ethylene-tetrafluoroethylene copolymer (ETFE), and a perfluoroalkoxy fluorine resin (PFA), an olefin-based resin such as polyethylene and polypropylene or a mixture thereof, polyurethane, polyester, polyamide, a polyether nylon resin, a mixture of the olefin-based resin and an ethylene-vinyl acetate copolymer, and the like.

A length of the catheter 12 is not particularly limited, and can be appropriately designed according to the application, various conditions, and the like, and is set to, for example, about 14 to 500 mm. The proximal end of the catheter 12 is inserted and fixed inside the catheter hub 20.

The catheter hub 20 is exposed on the patient's skin in a state in which the catheter 12 has been inserted into the patient's blood vessel, and indwells together with the catheter 12 by being pasted with a tape or the like. A material forming the catheter hub 20 is not particularly limited, but, for example, the materials exemplified in the needle hub 16 may be appropriately adopted.

As illustrated in FIGS. 1 and 2, the catheter hub 20 is formed into a tubular shape tapered in the distal direction. A hollow portion 30 is provided inside the catheter hub 20. The hollow portion 30 holds the catheter 12 on the distal side and communicates with the lumen 12a (proximal opening 12a2). In addition, the hollow portion 30 communicates with the proximal opening portion 30aof the catheter hub 20 on the proximal side. A flange 32 that protrudes to the radially outer side and extends along the circumferential direction is provided on the outer peripheral surface of the catheter hub 20 on the proximal side.

The catheter 12 and the catheter hub 20 are fixed by an appropriate fixing means such as caulking, fusion, or adhesion. In FIG. 2, a caulking pin 34 is inserted into the hollow portion 30 of the catheter hub 20 to caulk the caulking pin 34 by sandwiching the catheter 12 between an inner wall 20aof the catheter hub 20 and the caulking pin 34, thereby fixing the catheter 12.

A valve body 40A is provided in the hollow portion 30 of the catheter hub 20. Therefore, the hollow portion 30 of the catheter hub 20 is divided into a distal-side space 36 and a proximal-side space 38 with the valve body 40A as a base point. The distal-side space 36 is formed in a space tapered in the distal direction in order to arrange a funnel portion of the caulking pin 34. The inner wall 20aof the catheter hub 20 forming the distal-side space 36 is provided with a locking convex portion 37 locking the caulking pin 34.

Meanwhile, an inner diameter of the proximal-side space 38 of the catheter hub 20 gradually increases in the proximal direction, and the inner wall 20aof the catheter hub 20 forming the proximal-side space 38 is configured into a luer taper shape into which the connector 100 of the medical device can be fitted. Incidentally, a connection structure between the catheter hub 20 and the connector 100 is not particularly limited, and a connection portion of the catheter hub 20 may be formed into various shapes corresponding to a predetermined standard of the medical device.

In addition, the inner wall 20aof the catheter hub 20 is provided with a fixing structure 39 that inhibits the entire valve body 40A from moving in the axial direction in a state in which the valve body 40A is arranged. The fixing structure 39 is configured of a stepped portion 39ain which the diameter of the hollow portion 30 is slightly reduced, and a locking convex portion 39bprotruding inward at a position away from the stepped portion 39atoward the proximal side.

The valve body 40A has a slit 42 that can be open and closed, and the inner needle 14 extends through the slit 42 to form the multi-structure needle 11 in the initial state of the catheter assembly 10A. In addition, the valve body 40A according to the present embodiment has a configuration in which the slit 42 can be open and closed after removal of the inner needle 14 without providing an opening member configured to open a valve body applied to a conventional catheter hub. Hereinafter, the valve body 40A will be specifically described.

The valve body 40A has a fixed portion 44 fixed to the inner wall 20aof the catheter hub 20 and a deformable portion 46 protruding from the fixed portion 44 in the distal direction. The fixed portion 44 is configured as an annular structure that protrudes to the radially outer side of the deformable portion 46 and has a sufficient thickness in the axial direction of the catheter hub 20. In this manner, the fixed portion 44 can also be called an annular portion. The valve body 40A is immovably fixed by sandwiching the fixed portion 44 between the stepped portion 39aof the fixing structure 39 and the locking convex portion 39b.

The deformable portion 46 is configured as a so-called duckbill valve. Specifically, the deformable portion 46 is formed into a cylindrical shape on the fixed portion 44 side and has a pair of inclined plate portions 48 that are close to each other in the distal direction. As a result, a valve space 50 that narrows in the distal direction is formed inside the valve body 40A including the fixed portion 44.

A distal end of the pair of inclined plate portions 48 forms an extending end 52 extending in the width direction. The slit 42 of the valve body 40A is configured of a front slit 42a, formed along the longitudinal direction of the extending end 52, and side slits 42b, formed in a cylindrical side portion 54 formed between the pair of inclined plate portions 48 from both ends of the extending end 52, when viewed from the front. The front slit 42a and the side slits 42b are continuous, and the side slits 42b extend to be parallel in the proximal direction from the extending end 52 to the distal end of the fixed portion 44.

The fixed portion 44, the pair of inclined plate portions 48, and the side portion 54 are formed in such a shape that allows insertion of the inner needle 14 through the slit 42 in the initial state of the catheter assembly 10A and self-closes the slit 42 with the removal of the inner needle 14 from the valve body 40A.

Then, the valve body 40A according to the present embodiment includes a pusher portion 56 protruding from inner surfaces of the pair of inclined plate portions 48 (and the extending end 52) in the proximal direction. The pusher portion 56 is continuous with the inner surfaces configuring the valve space 50 of the deformable portion 46. That is, the valve body 40A is configured as an monolithic structure in which the fixed portion 44, the deformable portion 46, and the pusher portion 56 are monolithicly molded. The pusher portion 56 can also be referred to as a cylindrical portion.

The pusher portion 56 is connected to a connecting portion 46a that is slightly closer to the fixed portion 44 than an intermediate position of the deformable portion 46 (the pair of inclined plate portions 48) in the axial direction. The pusher portion 56 has a base 58 forming a basic skeleton of the pusher portion 56 and a plurality of ribs 60 provided on an outer peripheral surface of the base 58.

The base 58 is formed into a cylindrical shape, and a tubular wall 58a extends linearly along a displacement direction of the pusher portion 56 (the axial direction of the catheter hub 20). The base 58 extends to the fixed portion 44 positioned on the radially outer side through a gap 62 (in a non-contact manner). Inside the base 58, a part of a valve flow path 64 of the valve body 40A through which a liquid can flow is formed. A base slit 59 is formed on the distal side of the base 58 so as to overlap the side slit 42b. The base slit 59 is continuous with the front slit 42a.

The valve flow path 64 of the valve body 40A is configured of the pusher portion 56 and the deformable portion 46 (the pair of inclined plate portions 48) on the distal side of the pusher portion 56. Therefore, the valve flow path 64 is formed in the pusher portion 56 to have a constant flow path cross-sectional area, and the flow path cross-sectional area is gradually reduced in the distal direction in the deformable portion 46.

The base 58 (pusher portion 56) protrudes from the connecting portion 46a of the deformable portion 46 to be sufficiently longer than a proximal end of the fixed portion 44. The total length of the base 58 is not particularly limited, but for example, may be set to be 1.5 times or more longer than an axial length of a valve main body portion 47 formed of the fixed portion 44 and the deformable portion 46. In the initial state, a proximal end of the base 58 in the present embodiment is arranged at a substantially intermediate position of the proximal-side space 38 in the axial direction.

An outer diameter and an inner diameter of the base 58 are set to be constant along the extending direction. That is, the thickness of the tubular wall 58a is constant along the extending direction, and is greater than the thickness of the deformable portion 46. As a result, the rigidity of the base 58 is higher than the rigidity of the deformable portion 46.

In addition, the plurality of ribs 60 are configured as protruding ridges that protrude shortly to the radially outer side from the outer peripheral surface of the base 58 and extend linearly along the extending direction of the base 58.

The amount of the rib 60 protruding to the radially outer side is shorter than an interval between the gaps 62. Therefore, each of the ribs 60 is not in contact with the fixed portion 44. The ribs 60 are arranged at equal intervals along the circumferential direction of the base 58. Each of the ribs 60 is formed to have substantially the same length as the total length of the base 58, and reinforces an axially extending posture of the base 58. Incidentally, the number and shapes of the ribs 60 are not particularly limited, and the pusher portion 56 does not necessarily have the ribs 60. A distal end of the rib 60 is connected to the deformable portion 46.

A material forming the valve body 40A is not particularly limited, but examples thereof include elastic materials such as polybutadiene, nitrile, and chloroprene synthetic rubber, natural rubber such as polyisoprene, thermoset elastomers such as urethane rubber, silicon rubber, and fluoro-rubber, thermoplastic elastomers, and other elastomers.

The catheter assembly 10A according to the present embodiment is basically configured as described above, and operations thereof will be described hereinafter.

As described above, the catheter assembly 10A is used at the time of constructing the inlet/outlet for the infusion, the blood transfusion, the blood sampling, and the like to the patient. The user grips and operates the needle hub 16 of the catheter assembly 10A in the initial state shown in FIG. 1 to puncture the patient with the multi-structure needle 11.

When the needle tip 14a of the inner needle 14 reaches the blood vessel, blood flows through the groove 24 of the inner needle 14 into the lumen 12a of the catheter 12. The user visually confirms this flashback of blood to confirm that the catheter 12 has secured the blood vessel. The flashback blood flows from the proximal opening 12a2 of the catheter 12 into the distal-side space 36 of the catheter hub 20. At this time, the slit 42 of the valve body 40A is open thinly by inserting the inner needle 14 therethrough to communicate with the valve flow path 64, and air on the distal side of the valve body 40A can be removed through this gap. In addition, the slit 42 having the thin opening can slow down the speed at which the blood leaks into the proximal-side space 38.

In the puncture state, the user advances the catheter 12 relative to the inner needle 14 to insert the catheter 12 into the blood vessel, and retracts the inner needle 14 with respect to the catheter 12 when the catheter 12 is inserted into the blood vessel to some extent. When the needle tip 14a of the inner needle 14 is removed from the valve body 40A at the time of retracting the inner needle 14, the deformable portion 46 elastically restores to close the slit 42. When the inner needle 14 is further retracted, the inner needle 14 is detached from the proximal opening portion 30aof the catheter hub 20. As a result, the operating body 18 is separated from the catheter indwelling body 22, and the catheter indwelling body 22 indwells in the patient.

In the indwelling state of the catheter indwelling body 22, the user inserts the connector 100 of the medical device (a tube of an infusion line or a blood transfusion line, a syringe, or the like) from the proximal opening portion 30aof the catheter hub 20 into the hollow portion 30 as illustrated in FIG. 4. When the connector 100 moves inside the proximal-side space 38 of the catheter hub 20 in the distal direction, its distal end surface comes into contact with a proximal end of the pusher portion 56 of the valve body 40A. As a result, the connector 100 presses the pusher portion 56 in the distal direction during insertion into the catheter hub 20.

Elastic deformation of the deformable portion 46 is caused according to the displacement of the pusher portion 56 in the distal direction while the extending shape of the pusher portion 56 is maintained. At this time, the pair of inclined plate portions 48 stretch in the distal direction with respect to the fixed portion 44 that is fixed, and are also elastically deformed in directions to be separated from each other and orthogonal to the extending direction of the slit 42 (the vertical direction in FIG. 4). As a result, the slit 42 is open in the vertical direction, and the valve flow path 64 of the valve body 40A and the distal-side space 36 communicate with each other through the slit 42. Then, the slit 42 is sufficiently open in a state in which an outer wall of the connector 100 is fitted (luer-locked) to the inner wall 20aof the catheter hub 20. The above-described deformation of the pair of inclined plate portions 48 has a relatively small so-called restoring force by which the inclined plate portions 48 tend to return to their original shape. Therefore, it is possible to provide room in setting of a fitting force between the connector 100 and the catheter hub 20.

Thus, the catheter indwelling body 22 causes the liquid (infusion solution or blood) flowing through a flow path 100aof the connector 100 to flow in the order of the valve flow path 64, the slit 42, and the distal-side space 36, and flows from the distal-side space 36 into the lumen 12a of the catheter 12. As a result, the liquid flowing in the catheter 12 is favorably administered to the patient.

In addition, the catheter indwelling body 22 detaches the connector 100 from the catheter hub 20 after the infusion or blood transfusion is stopped. When the connector 100 is retracted inside the catheter hub 20, a pressing force is weakened so that the deformable portion 46 is elastically restored, and the pusher portion 56 is displaced in the proximal direction. That is, the deformable portion 46 brings the pair of inclined plate portions 48 adjacent to each other to close the slit 42. Therefore, the valve body 40A blocks between the distal-side space 36 and the valve flow path 64 again to inhibit the liquid in the distal-side space 36 from leaking to the proximal side.

In addition, the catheter indwelling body 22 can open the valve body 40A by the same operation as described above when an infusion or a blood transfusion is performed again.

That is, the catheter assembly 10A of the present embodiment can perform a plurality of infusions and blood transfusions by opening and closing the valve body 40A.

Incidentally, the present invention is not limited to the above-described embodiment, and various modifications can be made in accordance with a gist of the invention. For example, the deformable portion 46 (valve main body portion 47) and the pusher portion 56 may be made of different materials and may be configured into a monolithic structure by insert molding, outsert molding, or the like. Therefore, in the valve body 40A, it is possible to apply a hard resin material or a metal material to the pusher portion 56 while applying an elastic material to the deformable portion 46. The deformable portion 46 and the pusher portion 56 may be monolithicly configured using the same material, and then, the fixed portion 44 may be configured into the monolithic structure by the above-described molding.

In addition, the pusher portion 56 of the valve body 40A is not limited to the above configuration, and may have various configurations in which the slit 42 of the deformable portion 46 can be open by pressing the connector 100 of the medical device. As an example, the distal side of the pusher portion 56 may be formed so as to branch into bifurcated arm portions 68 connected to the pair of inclined plate portions 48 by providing a pair of notches 66 at the same position of the slit 42 as indicated by an alternate long and two short dashes line in FIG. 2. The bifurcated (pair of) arm portions 68 are curved outward so as to be separated from each other when the pusher portion 56 moves in the distal direction, and thus, can deform the pair of inclined plate portions 48 to expand the slit 42 widely.

In addition, the pusher portion 56 (base 58) is not limited to the cylindrical shape, and may be configured into a square tube shape or the like. In addition, the pusher portion 56 (base 58) may be configured as a plurality of plates, rods, mesh bodies or the like extending to be parallel in the axial direction of the catheter hub 20. In short, the pusher portion 56 can have various shapes as long as forming the basic skeleton capable of transmitting the pressing force, generated at the time of inserting the connector 100, to the deformable portion 46. That is, the pusher portion 56 can also be called a pressing force transmitting means.

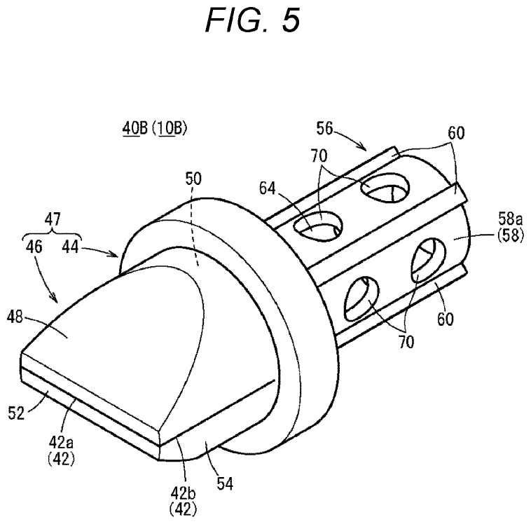

Hereinafter, some embodiments will be described in detail with reference to FIGS. 5 to 10B. Incidentally, an element having the same configuration or the same function as that in the above-described embodiment will be denoted by the same reference sign, and the detailed description thereof will be omitted in the following description.

### Second Embodiment

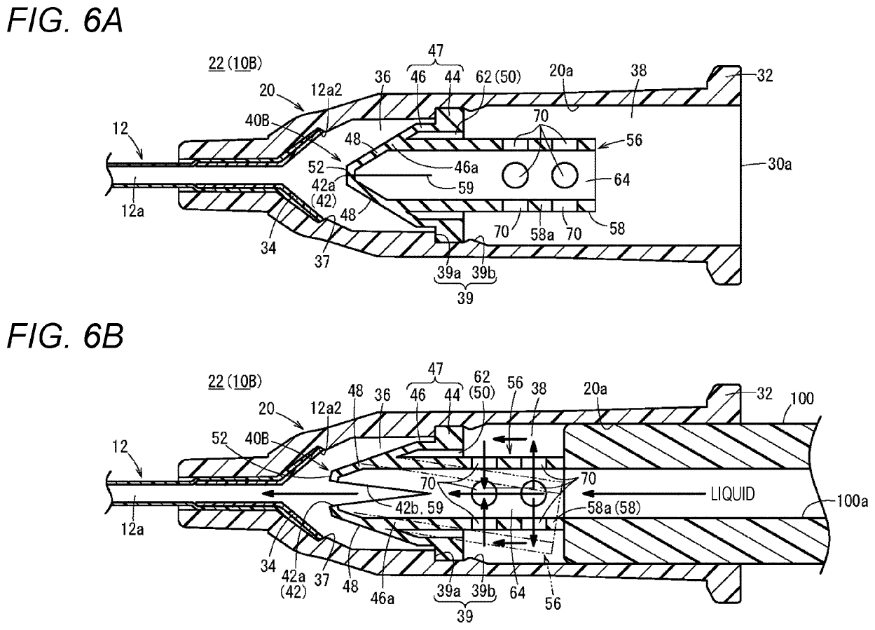

As illustrated in FIGS. 5, 6A, and 6B, a catheter assembly 10B according to a second embodiment is different from the above catheter assembly 10A in terms of including a valve body 40B in which a plurality of openings 70 are formed in the pusher portion 56.

Specifically, the pusher portion 56 is provided with the plurality of openings 70 so as to penetrate in the thickness direction on the tubular wall 58a of the cylindrical base 58 where the rib 60 is not provided. Each of the openings 70 is formed into a circular shape having a diameter smaller than a diameter of the valve flow path 64. Each of the openings 70 causes the valve flow path 64 inside the pusher portion 56 to communicate with the proximal-side space 38 outside the pusher portion 56.

The plurality of openings 70 are provided to be closer to the proximal side than an intermediate position of the base 58 in the extending direction. The respective openings 70 configure a plurality of (two) rows of openings along the extending direction of the base 58, and the rows of openings are arranged around the base 58 at intervals of 90° in the circumferential direction. Of course, the shapes, arrangement positions, number, and the like of the openings 70 can be appropriately designed.

Even when having the plurality of openings 70 as described above, the base 58 has the cylindrical basic skeleton and the plurality of ribs 60, and thus, has rigidity that is not elastically deformed (bent) along a displacement direction at the time of pressing the connector 100. Incidentally, the rigidity of the pusher portion 56 (tubular wall 58a) may be secured by setting the thickness of the pusher portion 56 (tubular wall 58a) to be greater than the thickness of the pusher portion 56 according to the first embodiment.

The catheter assembly 10B having the above valve body 40B pushes the pusher portion 56 in the distal direction at the time of inserting the connector 100 from the proximal opening portion 30aof the catheter hub 20, thereby elastically deforming the deformable portion 46 to open the slit 42. Therefore, the catheter assembly 10B according to the second embodiment can obtain the same effects as the catheter assembly 10A.

In particular, the catheter assembly 10B can cause a liquid flowing through the valve flow path 64 to flow out to the proximal-side space 38 through the opening 70, and cause the liquid to flow from the proximal-side space 38 into the valve flow path 64. For example, when the catheter assembly 10B is in a posture that the pusher portion 56 is inclined with respect to the connector 100 as indicated by an alternate long and two short dashes line in FIG. 6B, the liquid flowing from the flow path 100aof the connector 100 sometimes flows directly into the proximal-side space 38. On the other hand, the valve body 40B can guide the liquid flowing out to the proximal-side space 38 to the valve flow path 64 by providing the opening 70 in the pusher portion 56.

### Third Embodiment

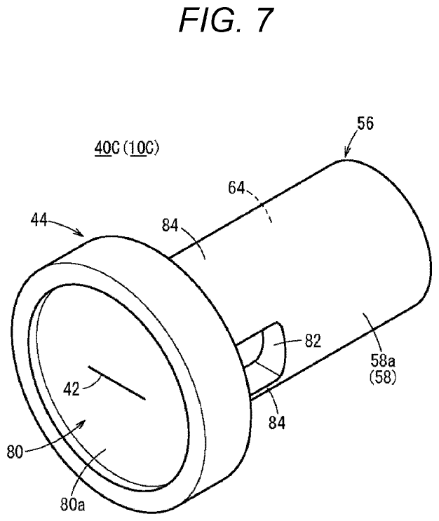

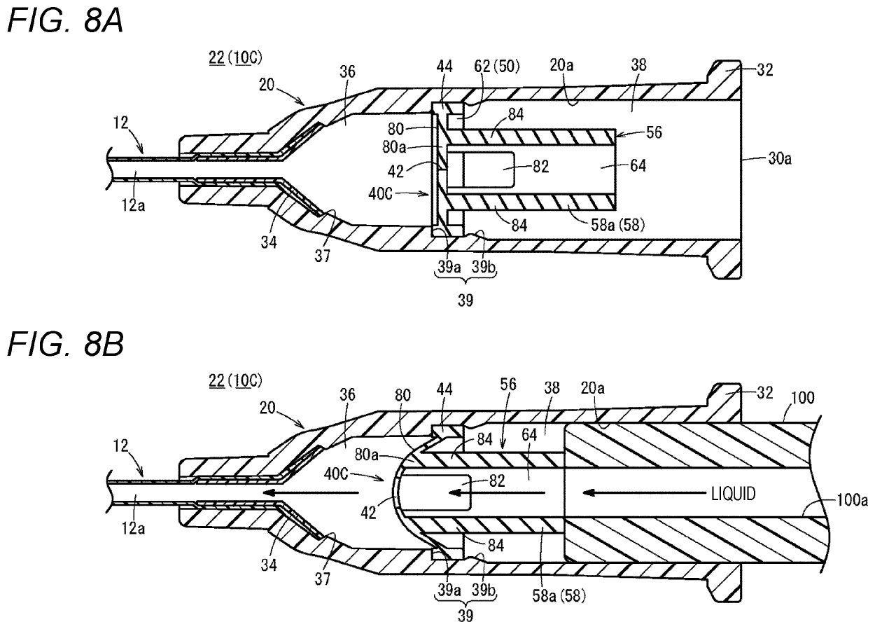

As illustrated in FIGS. 7, 8A, and 8B, a catheter assembly 10C according to a third embodiment is different from the above catheter assemblies 10A and 10B in terms of including a valve body 40C having a film-like deformable portion 80 inside the fixed portion 44.

Specifically, the deformable portion 80 is connected to an inner peripheral surface of the annular fixed portion 44, is formed into a disk shape protruding radially inward, and has the slit 42 extending in the width direction at the center thereof. A film wall 80aconfiguring the deformable portion 80 forms a flat shape in a state of not receiving a pressing force from the pusher portion 56, and the slit 42 is closed in this flat state. In addition, the thickness of the film wall 80aof the deformable portion 80 is formed to be thinner than the thickness of the tubular wall 58a of the pusher portion 56.

Meanwhile, the pusher portion 56 is connected to a surface of the deformable portion 80 facing the proximal direction. The pusher portion 56 is formed by the cylindrical base 58, and the base 58 is not provided with the rib 60. In addition, a pair of notches 82 opposing each other across the axial center of the base 58 are formed on the distal side of the base 58, and the pair of notches 82 extend from a proximal surface of the deformable portion 80 in a predetermined length (about ½ the axial length of the pusher portion 56) in the proximal direction. The pair of notches 82 have the same function as the opening 70 of the second embodiment for causing flow of a liquid. With the pair of notches 82, the distal side of the base 58 is configured as bifurcated (a pair of) arm portions 84 extending with the axial center of the base 58 interposed therebetween.

The pair of arm portions 84 are orthogonal to the extending direction of the slit 42 and are connected to the film wall 80aat a position slightly distant from the slit 42. Each of the arm portions 84 is formed into an arc shape in a cross-sectional view orthogonal to the axial direction of the base 58, and enhances the rigidity of the distal side of the base 58.

The catheter assembly 10C having the above valve body 40C pushes the pusher portion 56 in the distal direction at the time of inserting the connector 100 from the proximal opening portion 30aof the catheter hub 20, thereby elastically deforming the deformable portion 80. At this time, the pair of arm portions 84 of the pusher portions 56 push the film wall 80ain the distal direction and the vertical direction to widely expand the slit 42. In addition, if the proximal-side space 38 of the catheter hub 20 communicates with the flow path 100aof the connector 100, the notch 82 of the base 58 can cause the liquid flowing in the proximal-side space 38 to flow into the valve flow path 64 (that is, has the same function as the opening 70 of the second embodiment). Therefore, the catheter assembly 10C according to the third embodiment can obtain the same effects as the catheter assembly 10B.

### Fourth Embodiment

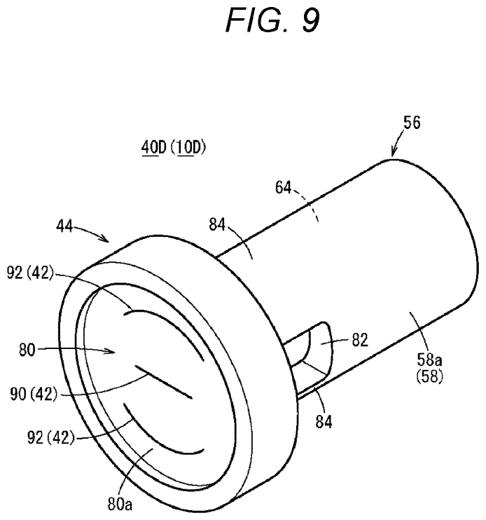

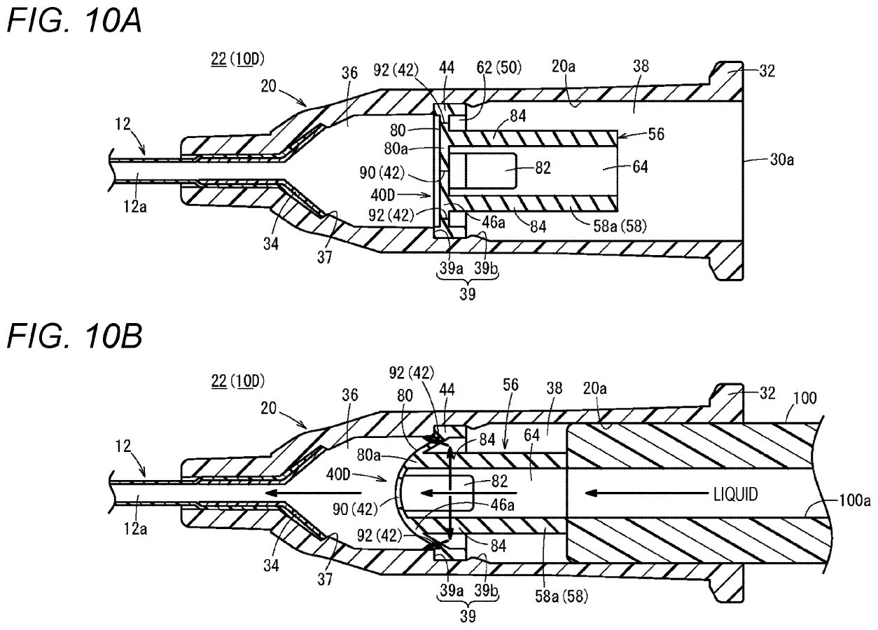

As illustrated in FIGS. 9, 10A, and 10B, a catheter assembly 10D according to a fourth embodiment is different from the above catheter assemblies 10A to 10C in terms of including a valve body 40D in which the plurality of slits 42 are formed in the deformable portion 80 according to the third embodiment.

Specifically, the plurality of slits 42 include a central slit 90 provided at the center of the disk-shaped deformable portion 80 and a pair of side slits 92 provided at positions separated by a predetermined distance in a direction orthogonal to the extending direction (width direction) of the central slit 90. That is, the deformable portion 80 of the present embodiment is provided with the three slits 42. The pair of side slits 92 are formed into an arc shape corresponding to the outer circumference of the deformable portion 80.

The pair of arm portions 84 of the pusher portion 56 are connected to the film wall 80abetween the central slit 90 and one side slit 92. Therefore, each of the side slits 92 is located between each of the pair of arm portions 84 and the fixed portion 44, and causes the distal-side space 36 of the catheter hub 20 to communicate with the proximal-side space 38 (the gap 62 of the pusher portion 56 of the fixed portion 44) in an open state.

The catheter assembly 10D having the above valve body 40D pushes the pusher portion 56 in the distal direction at the time of inserting the connector 100 from the proximal opening portion 30aof the catheter hub 20, thereby elastically deforming the deformable portion 80. At this time, the pair of arm portions 84 of the pusher portion 56 push the film wall 80ain the distal direction and the vertical direction to open each of the three slits 42.

Therefore, a liquid flowing from the flow path 100aof the connector 100 to the valve flow path 64 flows into the distal-side space 36 through the central slit 90. In addition, the notch 82 of the base 58 causes the liquid to flow out from the valve flow path 64 into the proximal-side space 38, and this liquid flows through the proximal-side space 38 sandwiched between the valve body 40D and the connector 100, and then, flows into the distal-side space 36 through each of the side slits 92. Incidentally, when the proximal-side space 38 of the catheter hub 20 communicates with the flow path 100aof the connector 100, the liquid flowing to the proximal-side space 38 can be made to flow through each of the side slits 92.

Therefore, the catheter assembly 10D according to the fourth embodiment can also obtain the same effects as the above-described catheter assemblies 10A to 10C. In particular, the liquid can be guided to the distal-side space 36 through the pair of side slits 92 even when flowing to the proximal-side space 38, and thus, the liquid can flow more smoothly.

Of course, the shapes, number, formation positions, and the like of the side slits 92 are not particularly limited. For example, it is sufficient to provide one or more side slits 92 in the deformable portion 80. In addition, for example, the side slit 92 can also be provided on the outer side along the width direction of the central slit 90, and may be configured to be open when the deformable portion 80 is elastically deformed in the distal direction. Further, the side slit 92 can also be formed in the valve body 40B of the second embodiment.

Technical ideas and effects that can be grasped from the above-described embodiments are described as follows.

In the catheter assemblies 10A to 10D, the deformable portions 46 and 80 are deformed and the slit 42 can be favorably open when the pusher portion 56 is displaced with the insertion of the connector 100 by the valve bodies 40A to 40D each of which is the monolithic structure of the fixed portion 44, the deformable portion 46 or 80, and the pusher portion 56. As a result, the liquid flowing from the connector 100 is smoothly guided to the distal side of the valve bodies 40A to 40D through the slit 42. Therefore, the catheter assemblies 10A to 10D can reduce the number of components with a simple configuration without requiring a conventional opening member, which makes it possible to reduce manufacturing time and manufacturing cost.

In addition, the pusher portion 56 includes the base 58 that extends in the displacement direction of the pusher portion 56 and has a flow passage (valve flow path 64) inside, and the thickness of the base 58 is greater than the thickness of each of the deformable portions 46 and 80. The catheter assemblies 10A to 10D can stably displace the base 58 along the distal direction at the time of inserting the connector 100 to elastically deform the deformable portions 46 and 80 since the thickness of the base 58 is greater than the thickness of each of the deformable portions 46 and 80.

In addition, the pusher portion 56 is formed into the tubular shape, and the tubular wall 58a of the pusher portion 56 is provided with an opening (the opening 70 or the notch 82) that enables the liquid to flow inside and outside the pusher portion 56. The catheter assemblies 10B to 10D cause the liquid in the connector 100 to flow inside and outside the pusher portion 56 through the opening (the opening 70 or the notch 82) so that the liquid is inhibited from staying in the proximal-side space 38 in the valve bodies 40B to 40D. In particular, when the liquid flows from the flow path 100aof the connector 100 to the proximal-side space 38, it is possible to return the liquid to the inside of the pusher portion 56 through the opening (the opening 70 or the notch 82).

In addition, the slit 42 includes a center-side slit (the slit 42 or the central slit 90) formed on the center side of a part (the connecting portion 46a) where the pusher portion 56 is connected to the deformable portion 80. The slit 42, closer to the center side than the connecting portion 46a, can be widely open when the deformable portion 80 is deformed with pushing of the pusher portion 56, so that the liquid flowing from the connector 100 can flow more smoothly.

In addition, the slit 42 includes the side slit 92 formed between the fixed portion 44 and the part where the pusher portion 56 is connected to the deformable portion 80. The side slit 92 formed between the fixed portion 44 and the pusher portion 56 communicates with the proximal-side space 38 of the valve body 40D. Accordingly, when the liquid flows from the flow path 100aof the connector 100 into the proximal-side space 38, the catheter assembly 10D can cause the liquid to flow into the distal-side space 36 of the valve body 40D through the side slit 92.

In addition, the pusher portion 56 has the pair of arm portions 84 on the distal side, and the pair of arm portions 84 are connected to the deformable portion 80 at the positions orthogonal to the extending direction of the slit 42. In the catheter assemblies 10C and 10D, the deformable portion 80 can be easily deformed in the direction orthogonal to the extending direction of the slit 42 to open the slit 42 at the time of inserting the connector 100 since the pair of arm portions 84 are connected to the deformable portion 80.

In addition, the pusher portion 56 has the rib 60 extending in the displacement direction of the pusher portion 56. In the valve bodies 40A and 40B, the rib 60 is provided in the pusher portion 56, and thus, the deformable portion 46 can be favorably deformed without crushing the pusher portion 56 when the connector 100 presses the pusher portion 56.

In addition, the deformable portion 46 has the pair of inclined plate portions 48 that protrude in the distal direction and are close to each other in the distal direction, and has the slit 42 at the distal end of the pair of inclined plate portions 48. In the valve bodies 40A and 40B, the opening and closing of the slit 42 can be easily switched by making the pair of inclined plate portions 48 close to each other or apart from each other.

In addition, the slit 42 is formed of the front slit 42aextending along the extending direction of the distal end and the side slit 42b that is continuous with the front slit 42aand extends along the side portion 54 connecting the pair of inclined plate portions 48. As a result, the valve bodies 40A and 40B can expand the slit 42 more widely, and the liquid can flow more smoothly.

In addition, the deformable portion 80 is configured of the film wall 80aextending in a flat shape from the fixed portion 44 to the radially inner side. In this manner, the catheter assemblies 10C and 10D can stably open the slit 42 even with the deformable portion 80 configured on the film wall 80ato enable the liquid to flow.

What is claimed is:

1. A catheter assembly comprising:
a catheter having a lumen;
a catheter hub located at a proximal end of the catheter and having a hollow portion that communicates with the lumen; and
a valve body located in the catheter hub, wherein the valve body is an monolithic structure comprising:
a fixed portion fixed to the catheter hub,
a deformable portion continuous with the fixed portion and having a slit that is openable and closable, and
a pusher portion extending from the deformable portion in a proximal direction,
wherein the pusher portion is configured to be pushed in a distal direction by a connector inserted into the hollow portion from a proximal end of the catheter hub to deform the deformable portion and open the slit.

2. The catheter assembly according to claim 1, wherein:
the pusher portion comprises a base extending in a displacement direction of the pusher portion and having a flow passage inside; and
a thickness of the base is greater than a thickness of the deformable portion.

3. The catheter assembly according to claim 1, wherein:
the pusher portion has a tubular shape, and a tubular wall of the pusher portion comprises an opening that enables liquid to flow into and out of the pusher portion.

4. The catheter assembly according to claim 1, wherein:
the slit includes a center-side slit formed on a center side of a part where the pusher portion is connected to the deformable portion.

5. The catheter assembly according to claim 1, wherein:
the slit includes a side slit formed between the fixed portion and the part where the pusher portion is connected to the deformable portion.

6. The catheter assembly according to claim 1, wherein:
the pusher portion comprises a pair of arm portions on a distal side; and
the pair of arm portions are connected to the deformable portion at positions orthogonal to an extending direction of the slit.

7. The catheter assembly according to claim 1, wherein:
the pusher portion comprises a rib extending in the displacement direction of the pusher portion.

8. The catheter assembly according to claim 1, wherein:
the deformable portion comprises a pair of inclined plate portions that protrude in the distal direction; and
the slit is located at a distal end of the pair of inclined plate portions.

9. The catheter assembly according to claim 8, wherein:
the slit is formed of a front slit extending along an extending direction of the distal end and a side slit that is continuous with the front slit and extends along a side portion connecting the pair of inclined plate portions.

10. The catheter assembly according to claim 1, wherein:
the deformable portion is formed of a flat wall extending from the fixed portion to a radially inner side.

11. A catheter assembly comprising:
a catheter having a lumen;
a catheter hub located at a proximal end of the catheter and having a hollow portion that communicates with the lumen; and
a valve body located in the catheter hub, wherein the valve body is an monolithic structure comprising:
a fixed portion fixed to the catheter hub, wherein the fixed portion is an annular structure,
a deformable portion continuous with the fixed portion and having a slit that is openable and closable,
wherein the deformable portion comprises a pair of inclined plate portions that protrude in the distal direction, and wherein the slit is located at a distal end of the pair of inclined plate portions, and
a pusher portion extending from the deformable portion in a proximal direction, wherein the pusher portion comprises a cylindrical base extending in a displacement direction of the pusher portion and having a flow passage inside,
wherein the pusher portion is configured to be pushed in a distal direction by a connector inserted into the hollow portion from a proximal end of the catheter hub to deform the deformable portion and open the slit.

12. A catheter assembly comprising:
a catheter having a lumen;
a catheter hub located at a proximal end of the catheter and having a hollow portion that communicates with the lumen; and
a valve body located in the catheter hub, wherein the valve body is an monolithic structure comprising:
a fixed portion fixed to the catheter hub, wherein the fixed portion is an annular structure,
a deformable portion continuous with the fixed portion and having a slit that is openable and closable, wherein the deformable portion is formed of a flat wall extending from the fixed portion to a radially inner side, and
a pusher portion extending from the deformable portion in a proximal direction, wherein the pusher portion comprises a cylindrical base extending in a displacement direction of the pusher portion and having a flow passage inside,
wherein the pusher portion is configured to be pushed in a distal direction by a connector inserted into the hollow portion from a proximal end of the catheter hub to deform the deformable portion and open the slit.
